(19) United States
(12) Patent Application Publication
(10) Pub. No.: US 2016/0339069 A1
(43) Pub. Date: Nov. 24, 2016

(54) METHOD FOR PREPARING MEDICINAL HERB COMPOSITION HAVING ATOPIC IMMUNE SYSTEM STRENGTHENING AND ANTICANCER EFFECTS

(71) Applicant: Gyeol GO, Busan (KR)

(72) Inventor: Gyeol GO, Busan (KR)

(21) Appl. No.: 15/114,516

(22) PCT Filed: Jan. 27, 2015
(86) PCT No.: PCT/KR2015/000861

§ 371 Date: Jul. 27, 2016

(30) Foreign Application Priority Data
Jan. 29, 2014 (KR) 10-2014-0011078

(51) Int. Cl. A61K 36/254; A61K 36/899; A61K 36/9068; A61K 36/54; A23C 9/133; A61K 36/752; A61K 9/00; C12P 1/04; A23C 9/13; A61K 36/78; A61K 36/725

(52) U.S. Cl. CPC A61K 36/254; A61K 36/78; A61K 36/899; A61K 36/9068; A61K 36/54; A61K 36/725; A61K 36/752; A61K 9/0053; C12P 1/04; A23C 9/1307; A23C 9/133; A61K 2236/13; A61K 2236/37; A61K 2236/51

(57) ABSTRACT

Disclosed is a method for preparing an herbal composition having atopic immune system strengthening and anticancer effects, wherein the herbal composition contains Siberian ginseng, fish mint, Manchurian wild rice, dried ginger, cinnamon bark, jujube, and citrus peel. The herbal composition, obtained by the method provided, is effective at scavenging reactive oxygen species (ROS), promoting the proliferation of normal cells without toxicity, and inhibiting the proliferation of colon cancer cells without serious toxicity, based on the results of tests conducted at the Blue-Bio Industry Regional Innovation Center, Dong-Eui University, Busan.

### TECHNICAL FIELD

The present invention relates to a method for preparing an herbal composition having atopic immune system strengthening and anticancer effects and, more particularly, to a method for preparing an herbal composition having atopic immune system strengthening and anticancer effects, wherein the herbal composition contains Siberian ginseng, fish mint, Manchurian wild rice, dried ginger, cinnamon bark, jujube, and citrus peel.

### BACKGROUND ART

Atopy is a term that was used for the first time by a scholar named Coca at 1925, and refers to a congenital allergic reaction to foods or inhaled substances. The term “atopy” is derived from the Greek word, meaning “unknown” or “oddly”. As can be seen from its meaning, atopy is an incurable disease, which is difficult to treat, and the exact cause thereof is not known. Atopic dermatitis is chronic dermatitis that mainly occurs in persons who are genetically prone to eczema, hay fever, asthma, allergies, etc. and is often called congenital fever, and atopic dermatitis starts from infant eczema and shows chronic and recurrent delayed progression.

The causes of atopy have not yet been identified with certainty, and are deemed to be associated with genetic and immunological factors and also with various peripheral factors, such as environmental, social, and psychological factors.

Atopic dermatitis therapy today includes external and internal methods, depending on the manner of drug administration, and immunotherapy. Examples of the external method may include cleaning the skin, applying a steroidal ointment, applying a moisturizer, etc. In particular, a steroid, which is a kind of adrenal cortical hormone, exhibits very good therapeutic effects but causes side effects upon long-term use and thus should be carefully used. Applying the moisturizer means that a cosmetic product for supplying moisture to dry skin and maintaining moisture is applied to dry skin.

Examples of an atopic dermatitis drug used for the internal method mainly include antihistamines and steroidal medicaments or herbal drugs. Immunotherapy is intended to naturally strengthen the ability of body's immune system under appropriate environment.

For the internal method, in the field of Oriental medicine it though that atopic dermatitis is caused because toxins are generated from heat accumulating in the body. Heat toxins deep inside the body may move up to the skin layer to thus cause various problems.

Unless such heat toxins are relieved, atopic dermatitis may inevitably recur. Particularly in the case where pregnant woman eats a large amount of spicy or salty foods or excessive caffeine, alcohol, fast foods, greasy foods, etc. or where the womb contains a lot of waste left behind therein or is weak, heat may accumulate in the womb and may then be transferred to the fetus to thus cause atopic dermatitis.

Heat toxins not only may incur skin abnormalities but may also deteriorate the ability of body's immune system to thus cause various complications. If the immune system is weakened, abnormal cells are expressed and may then progress to cancer, and thus, not only should the symptoms be treated, but the basic causes of atopic dermatitis must also be found and solved. Hence, the immune system needs to be strengthened so as to prevent the activation of cancer cells upon the collapse thereof, whereby atopic dermatitis may be naturally treated, and moreover, anticancer effects may be exhibited.

Conventional techniques pertaining to atopy include an herbal composition for the treatment of atopy, a composition for the amelioration of atopic dermatitis, and a cosmetic composition for ameliorating atopic dermatitis symptoms using Orostachys japonica.

However, since the above compositions are incapable of performing essential treatment through immune system strengthening and may exhibit limited effects, there is urgently required a novel herbal composition, which has small side effects and may strengthen the immune system to thus enable the essential treatment and which may also alleviate atopy and manifest anticancer effects.

### DISCLOSURE

### Technical Problem

Accordingly, the present invention has been made keeping in mind the above problems encountered in the related art, and the present invention is intended to provide a method for preparing an herbal composition having atopic immune system strengthening and anticancer effects, suitable for solving atopic problems based on the prescription principle of traditional medicines, wherein the herbal composition contains Siberian ginseng, fish mint, Manchurian wild rice, dried ginger, cinnamon bark, jujube and citrus peel.

### Technical Solution

Therefore, the present invention preferably provides a method for preparing an herbal composition having atopic immune system strengthening and anticancer effects, including the following steps of:

(a) providing an herbal mixture including Siberian ginseng, fish mint, Manchurian wild rice, dried ginger, cinnamon bark, jujube and citrus peel through water washing; and

(b) adding the herbal mixture with water at a ratio of 1:9 to 12 and heating the herbal mixture at 100 to 120° C. for 4 to 5 hr so as to carry out hydrothermal extraction.

As such, the herbal mixture is preferably composed of 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel.

Also, the present invention preferably provides a method for preparing an herbal composition having atopic immune system strengthening and anticancer effects, which further includes, in addition to the above steps, an acquisition step, the method including the following steps of:

(a) providing an herbal mixture including Siberian ginseng, fish mint, Manchurian wild rice, dried ginger, cinnamon bark, jujube and citrus peel through water washing;

(b) adding the herbal mixture with water at a ratio of 1:9 to 12 and heating the herbal mixture at 100 to 120° C. for 4 to 5 hr so as to carry out hydrothermal extraction; and

(c) concentrating the hydrothermal extract in a vacuum at 70 to 75° C., thus obtaining a distillate.

Also, the present invention preferably provides a method for preparing an herbal composition having atopic immune system strengthening and anticancer effects, which further includes, in addition to the above steps, some steps, the method including the following steps of:

(a) providing an herbal mixture including Siberian ginseng, fish mint, Manchurian wild rice, dried ginger, cinnamon bark, jujube and citrus peel through water washing;

(b) adding the herbal mixture with water at a ratio of 1:9 to 12 and heating the herbal mixture at 100 to 120° C. for 4 to 5 hr so as to carry out hydrothermal extraction;

(c) concentrating the hydrothermal extract in a vacuum at 70 to 75° C., thus obtaining a distillate;

(d) mixing the distillate with milk at a ratio of 1:3 to give a mixture that is then fermented using lactobacillus, thus preparing a yogurt;

(e) adding blueberry and sugar at a ratio of 1:1 with a predetermined amount of water and performing heating for 30 min to 1 hr, thus preparing a jam; and

(f) adding the yogurt of (d) with the jam of (e), thus yielding a sweet yogurt.

### Advantageous Effects

According to the present invention, an herbal composition, obtained by the method for preparing an herbal composition having atopic immune system strengthening and anticancer effects, is effective at scavenging reactive oxygen species (ROS), promoting the proliferation of normal cells without toxicity, and inhibiting the proliferation of colon cancer cells without serious toxicity, based on the results of tests conducted at the Blue-Bio Industry Regional Innovation Center, Dong-Eui University, Busan.

### DESCRIPTION OF DRAWINGS

FIG. 1 is a flowchart showing a process of preparing an herbal composition having atopic immune system strengthening and anticancer effects according to a first embodiment of the present invention;

FIG. 2 is a flowchart showing a process of preparing an herbal composition having atopic immune system strengthening and anticancer effects according to a second embodiment of the present invention;

FIG. 3 is a flowchart showing a process of preparing an herbal composition having atopic immune system strengthening and anticancer effects according to a third embodiment of the present invention;

FIG. 4 is a graph showing the ROS scavenging activity of an herbal composition (2 Brix) according to a first embodiment of the present invention;

FIG. 5 is a graph showing the ROS scavenging activity of an herbal composition (8 Brix) according to a second embodiment of the present invention;

FIG. 6 is a graph showing the cell viability of a normal control group using the herbal composition (2 Brix) according to the first embodiment of the present invention;

FIG. 7 is a graph showing the cell viability of a normal control group using the herbal composition (8 Brix) according to the second embodiment of the present invention; and

FIG. 8 is a graph showing the cell viability of human colon cancer cells using the herbal composition (8 Brix) according to the second embodiment of the present invention.

### BEST MODE

Hereinafter, a detailed description will be given of a method for preparing an herbal composition having atopic immune system strengthening and anticancer effects according to embodiments of the present invention, with reference to the appended drawings.

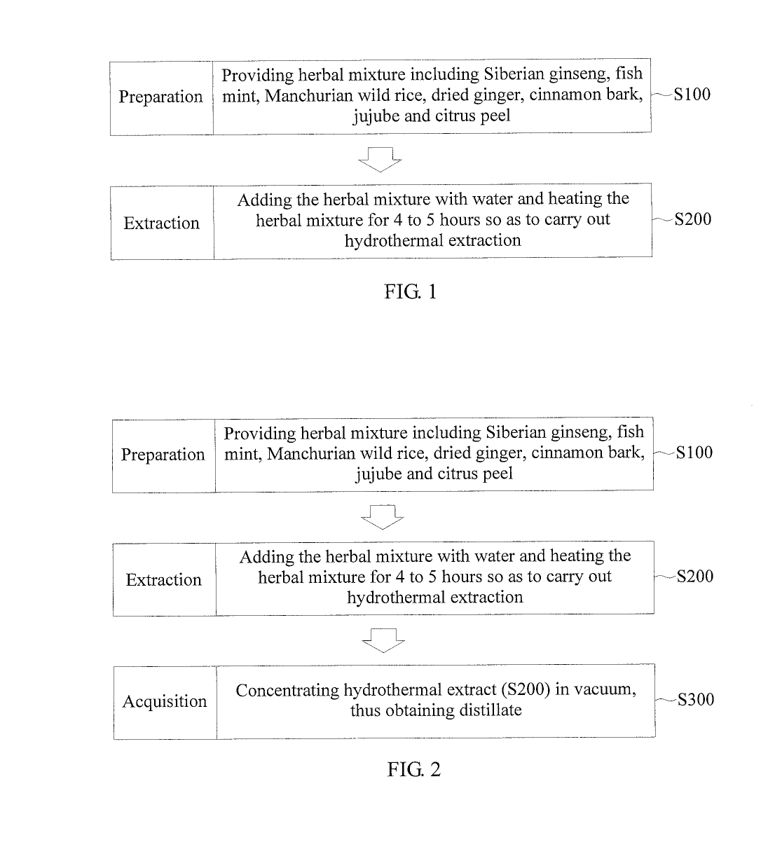

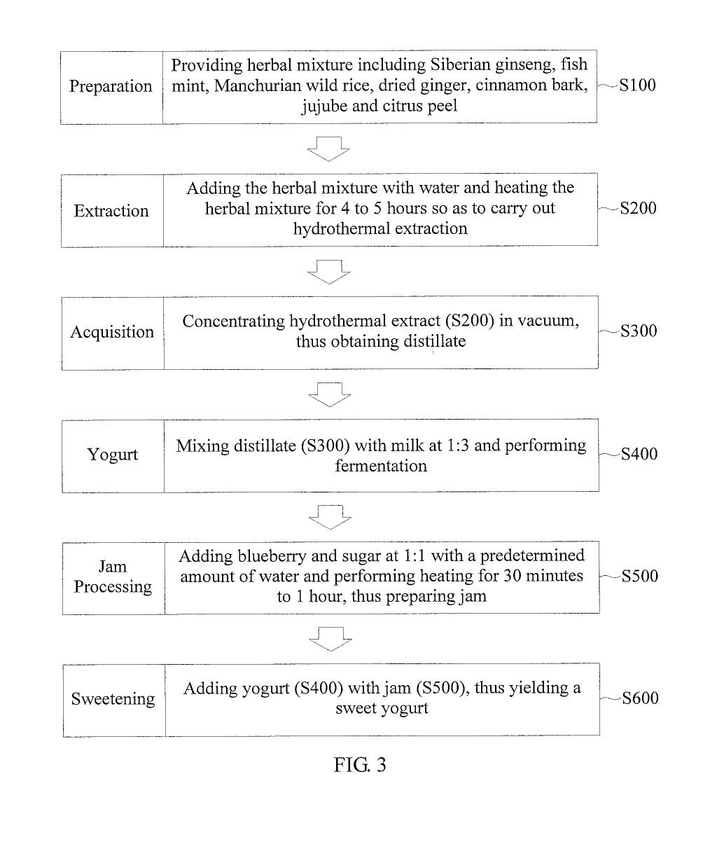

FIG. 1 is a flowchart showing a process of preparing an herbal composition having atopic immune system strengthening and anticancer effects according to a first embodiment of the present invention, FIG. 2 is a flowchart showing a process of preparing an herbal composition having atopic immune system strengthening and anticancer effects according to a second embodiment of the present invention, and FIG. 3 is a flowchart showing a process of preparing an herbal composition having atopic immune system strengthening and anticancer effects according to a third embodiment of the present invention.

As shown in FIG. 1, the method for preparing an herbal composition having atopic immune system strengthening and anticancer effects according to the first embodiment of the present invention includes providing an herbal mixture including Siberian ginseng, fish mint, Manchurian wild rice, dried ginger, cinnamon bark, jujube and citrus peel through water washing (S100) and adding the herbal mixture with water at a ratio of 1:9 to 12 and heating the herbal mixture at 100 to 120° C. for 4 to 5 hr so as to carry out hydrothermal extraction (S200).

The herbal mixture is preferably composed of 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel. More specifically, very good effects may be exhibited when the herbal mixture is configured to include 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube, and 17 parts by weight of citrus peel.

However, depending on the individual constitutions of persons, the amounts of some components of the herbal mixture may fall out of the above ranges, which may be easily inferred by those skilled in the art to which the present invention applies, and the use range of the herbal mixture is dependent on the results of years of research based on traditional medicine, and the herbal mixture is effective at strengthening the atopic immune system and treating cancer, and may manifest optical effects in the above use range.

In order to maximally ensure the atopic immune system strengthening and anticancer effects, the herbal composition may be appropriately constituted by increasing or decreasing the amounts by weight of the components of the herbal mixture.

As shown in FIG. 2, the method for preparing an herbal composition according to the second embodiment of the present invention includes providing an herbal mixture including Siberian ginseng, fish mint, Manchurian wild rice, dried ginger, cinnamon bark, jujube and citrus peel through water washing (S100), adding the herbal mixture with water at a ratio of 1:9 to 12 and heating the herbal mixture at 100 to 120° C. for 4 to 5 hr so as to carry out hydrothermal extraction (S200), and concentrating the hydrothermal extract in a vacuum at 70 to 75° C., thus obtaining a distillate (S300).

The herbal mixture is preferably composed of 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel. More specifically, very good effects may be exhibited when the herbal mixture is configured to include 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube, and 17 parts by weight of citrus peel.

As shown in FIG. 3, the method for preparing an herbal composition according to the third embodiment of the present invention includes providing an herbal mixture including Siberian ginseng, fish mint, Manchurian wild rice, dried ginger, cinnamon bark, jujube and citrus peel through water washing (S100), adding the herbal mixture with water at a ratio of 1:9 to 12 and heating the herbal mixture at 100 to 120° C. for 4 to 5 hr so as to carry out hydrothermal extraction (S200), concentrating the hydrothermal extract in a vacuum at 70 to 75° C., thus obtaining a distillate (S300), mixing the distillate with milk at a ratio of 1:3 to give a mixture that is then fermented using lactobacillus, thus preparing a yogurt (S400), adding blueberry and sugar at a ratio of 1:1 with a predetermined amount of water and performing heating for 30 min to 1 hr, thus preparing a jam (S500), and adding the yogurt of S400 with the jam of S500, thus yielding a sweet yogurt (S600).

The herbal mixture is preferably composed of 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel. More specifically, very good effects may be exhibited when the herbal mixture is configured to include 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube, and 17 parts by weight of citrus peel.

Atopic dermatitis, which is caused by various factors, is mainly induced by the abnormal reaction or over-reaction of the immune system. Free radicals are used as the main defense means in the immune system and perform a positive function such as lysis of bacteria penetrating living organisms from outside. In patients with atopic dermatitis, however, ROS is excessively produced with respect to external stimuli to thus destroy the skin moisturizing layer, so that the skin becomes dried, and thus, ROS acts as a pathological factor that shows an immune response sensitive to external stimuli. Also, in patients with atopic dermatitis, a variety of antioxidant systems for decomposing ROS are known to be decreased. Hence, the herbal composition according to the present invention exhibits an antioxidant capacity, and whether or not atopic dermatitis may be ameliorated may be determined based on the ROS scavenging activity.

Unusual microbial transition, which occurs on the skin of atopic patients, causes atopic skin disease to become chronic, and is thus regarded as an important factor that gives rise to chronic atopic dermatitis. Atopic dermatitis is characterized in that the inherent microbial control capacity of the skin may be remarkably decreased and microbial transition on the skin of normal persons is considerably altered. A natural human antibiotic material, namely a peptide material, such as beta-defensin or cathelicidin, is secreted from the skin of normal persons, but the expression thereof is remarkably decreased from the skin of patients with atopic dermatitis. Also, an atopic pathogen, namely Staphylococcus aureus, an enzyme, namely Malassezia furfur, etc., which show almost none of microbial transition on the skin of normal persons, are reported to occur at high ratios. Thus, whether the herbal composition for the amelioration of atopy according to the present invention may exhibit bacteriostatic action against bacteria and fungi even without preservative treatment may be checked.

A better understanding of the present invention may be obtained through the following examples, which are set forth to illustrate, but are not to be construed as limiting the scope of the present invention, as will be apparent to those skilled in the art.

In particular, only the preparation method using components in the amounts shown in Table 1 below is described in the following examples, but mixing 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel may be easily performed by those skilled in the art.

### EXAMPLE 1

### Preparation of Herbal Composition of the Present Invention

In the present invention, an herbal mixture is composed of seven components, and the herb names, scientific names and amounts thereof are given in Table 1 below.

TABLE 1

| Herb name | Scientific name and usable portion | Amount (%) |
|---|---|---|
| Siberian ginseng | Acanthopanax senticosus root | 17 |
| Fish mint | Houttuynia cordata | 17 |
| Manchurian wild | Zizania latifolia | 8 |
| rice | | |
| Dried ginger | Zingiber officinale Roscoe | 17 |
| Cinnamon bark | Cinnamomum cassia Blume | 8 |
| Jujube | Zizyphus jujuba Mill var. | 16 |
| | inermis Rehder | |
| Citrus peel | Fraxinus rhynchophylla Hance | 17 |

In order to obtain the herbal composition having atopic immune system strengthening and anticancer effects according to embodiments of the present invention, the herbal composition was prepared using components in the amounts shown in Table 1 through the following three methods.

As shown in FIG. 1 regarding the method according to a first embodiment of the present invention, an herbal composition was obtained by providing an herbal mixture including 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube and 17 parts by weight of citrus peel through water washing, and adding the herbal mixture with water at a ratio of 1:9 to 12 and heating the herbal mixture at 100 to 120° C. for 4 to 5 hr so as to carry out hydrothermal extraction, thus yielding a hydrothermal extract.

The obtained herbal composition is a liquid with a concentration of about 2 Brix.

As shown in FIG. 2 regarding the method according to a second embodiment of the present invention, an herbal composition was obtained by providing an herbal mixture including 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube and 17 parts by weight of citrus peel through water washing, adding the herbal mixture with water at a ratio of 1:9 to 12 and heating the herbal mixture at 100 to 120° C. for 4 to 5 hr to carry out hydrothermal extraction, and concentrating the hydrothermal extract at 70 to 75° C. in a vacuum, thus yielding a distillate.

The obtained herbal composition is a liquid with a concentration of about 8 Brix.

As shown in FIG. 3 regarding the method according to a third embodiment of the present invention, an herbal composition was obtained by providing an herbal mixture including 17 parts by weight of Siberian ginseng, 17 parts by weight of fish mint, 8 parts by weight of Manchurian wild rice, 17 parts by weight of dried ginger, 8 parts by weight of cinnamon bark, 16 parts by weight of jujube and 17 parts by weight of citrus peel through water washing, adding the herbal mixture with water at a ratio of 1:9 to 12 and heating the herbal mixture at 100 to 120° C. for 4 to 5 hr so as to carry out hydrothermal extraction, concentrating the hydrothermal extract at 70 to 75° C. in a vacuum, thus obtaining a distillate, mixing the distillate with milk at a ratio of 1:3 to give a mixture that was then fermented using lactobacillus, thus preparing a yogurt, adding blueberry and sugar at a ratio of 1:1 with a predetermined amount of water and performing heating for 30 min to 1 hr, thus preparing a jam, and adding the jam to the yogurt, thus yielding a sweet yogurt.

In the present invention, all of the herbal compositions obtained by the above three methods are provided in liquid phase. The herbal composition obtained by the method according to the first embodiment of the present invention has a concentration of 2 Brix, the herbal composition obtained by the method according to the second embodiment of the present invention has a concentration of 8 Brix, and the herbal composition obtained by the method according to the third embodiment of the present invention is provided in the form of a sweet yogurt which may be easily taken by general persons.

### EXAMPLE 2

### Analysis of Herbal Composition of the Present Invention

The herbal composition (2 Brix) obtained by the method according to the first embodiment of the present invention and the herbal composition (8 Brix) obtained by the method according to the second embodiment of the present invention were analyzed for ROS scavenging activity and cell viability at the Blue-Bio Industry Regional Innovation Center, Dong-Eui University, Busan.

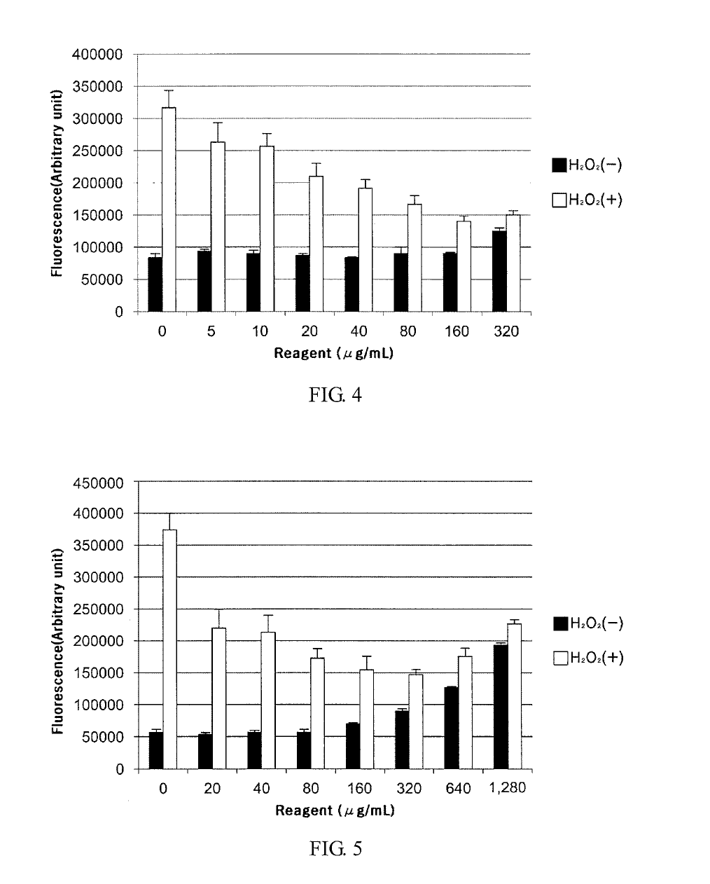

FIG. 4 is a graph showing the results of analysis of the ROS scavenging activity of the herbal composition (2 Brix) according to the first embodiment of the present invention, and FIG. 5 is a graph showing the results of analysis of the ROS scavenging activity of the herbal composition (8 Brix) according to the second embodiment of the present invention.

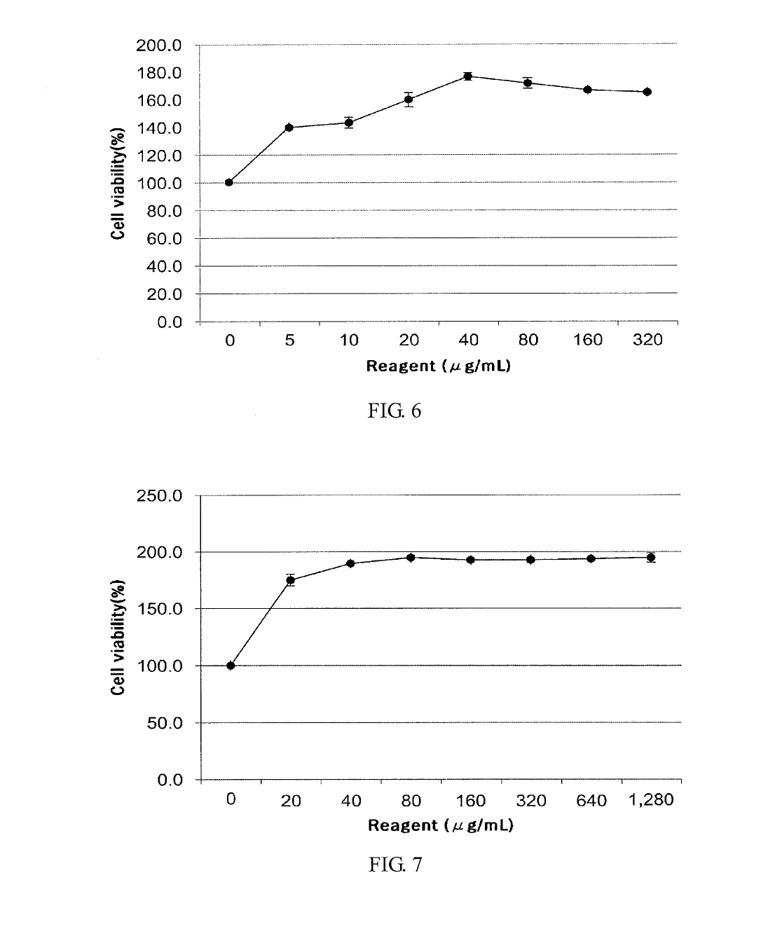

FIG. 6 is a graph showing the results of analysis of the effect of the herbal composition (2 Brix) according to the first embodiment of the present invention on the cell proliferation of a normal control group RAW 264.7, and FIG. 7 is a graph showing the results of analysis of the effect of the herbal composition (8 Brix) according to the second embodiment of the present invention on the cell proliferation of a normal control group RAW 264.7.

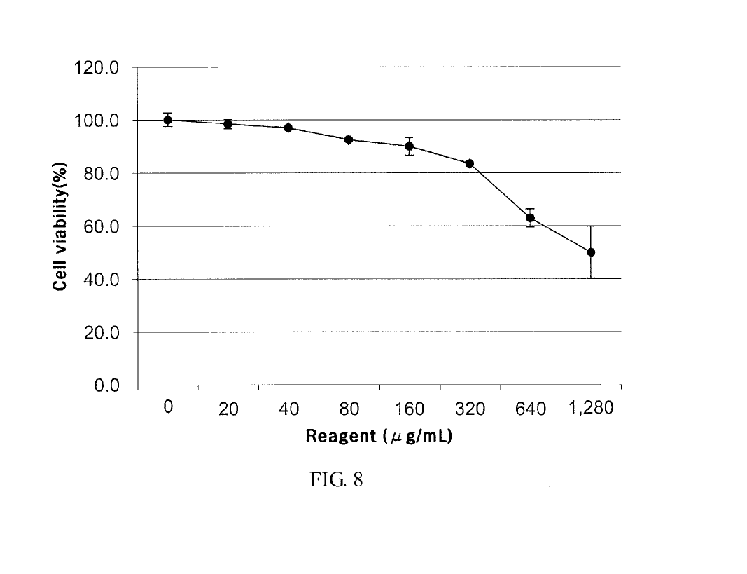

FIG. 8 is a graph showing the results of analysis of the effect of the herbal composition (8 Brix) according to the second embodiment of the present invention on the proliferation of a human colon cancer cell line HT-29.

The following analysis summary, analysis method, analysis results, and conclusion are based on reports from the Blue-Bio Industry Regional Innovation Center, Dong-Eui University, Busan.

1. Analysis summary: Analysis of effects of test samples on ROS scavenging activity and on cell viability of two kinds of cell lines

2. Analysis Method

1) Preparation of samples: test samples (a liquid sample at 2 Brix and a liquid sample at 8 Brix) were refrigerated at 4 before use, and used for tests by being sterilized through heating at 100° C. for 15 min and then rapidly cooled using ice.

2) Culture of cell line: Two cell lines used for tests, namely RAW 264.7 and HT-29, were purchased from the American Type Culture Collection (ATCC), USA, and cultured in a DMEM medium containing 10% fetal bovine serum (FBS) and penicillin/streptomycin and then used for tests.

3) Measurement of ROS scavenging activity: The ROS scavenging activity of the samples was measured using a mouse macrophage cell line, namely a RAW 264.7 cell line.

Using hydrogen peroxide (H₂O₂) as a typical source of ROS, the effect of the sample on the production of ROS by H₂O₂ was measured. Specifically, RAW 264.7 cells were pretreated with, as a cell permeable fluorescent dye, 50 μM 2′,7′-dichlorofluorescin diacetate (DCFH-DA) for 2 hr, and then treated with 500 μM H₂O₂ and the samples at different concentrations, after which the ROS scavenging activity of the samples was evaluated through fluorescence measurement using a multiplate reader. The measurement was performed three times and the measured values were then averaged, and the results were represented by mean±standard deviation (SD).

4) Measurement of cell viability: The effects of the samples on the cell proliferation and survival of a normal control group RAW 264.7 and a human colon cancer cell line HT-29 were measured through a water-soluble tetrazolium (WST) assay. Each cell line was allowed to attach for 24 hr through seeding of 1×10⁵ cells per well to a 24-well tissue culture plate, and was treated with the samples at different concentrations for 24 hr or 48 hr, after which the effects of the samples on cell viability were measured through WST assay. The measurement was performed three times, the measured values were then averaged, and the results were represented by mean ±standard deviation (SD).

3. Analysis Results

1) ROS scavenging activity: After treatment with 500 μM H₂O₂ and samples at different concentrations, the ROS inhibitory activity of the samples was determined by measuring the fluorescence amount.

As shown in FIGS. 4 and 5, the cells were treated with the 2 Brix and 8 Brix samples at different concentrations, and then the ROS scavenging activity of the samples was measured. As results, concentration-dependent ROS inhibitory activity, namely concentration-dependent ROS scavenging activity, was observed through treatment with the samples at 5, 10, 20, 40, 80, and 160 μg/ml, and the ROS generated by the sample itself was not observed.

Upon treatment with the samples at 320, 640, and 1,280 μg/ml, the decreased ROS was increased again, and there was a likelihood of producing ROS upon treatment with a high-concentration sample.

2) Cell viability: The effects of the samples on the cell proliferation and survival of a normal control group, namely a mouse macrophage cell line RAW 264.7, and a human colon cancer cell line HT-29, were measured through WST assay. The effects of the samples on cell proliferation of a normal control group, i.e. the RAW 264.7 cell line, were first analyzed.

As shown in FIGS. 6 and 7, no toxicity was exhibited in the corresponding cell line upon treatment with the samples at concentrations of 5 to 320 μg/ml and 20 to 1,280 μg/ml, and the cell proliferation rate was further increased through sample treatment. These samples were determined to show no toxicity for the RAW 264.7 cell line used in this test.

Also, the effect of the sample on the proliferation of a human colon cancer cell line HT-29 was measured. As shown in FIG. 8, severe changes in cytotoxicity and cell morphology were not observed using a microscope, and the cell proliferation rate was decreased with an increment in the concentration of the sample in the concentration range of 20 to 1,280 μg/ml. When the cell viability of the group not treated with the sample was set to 100%, the results of cell viability were 98.3, 97.1, 93.1, 90.7, 82.8, 62.6, and 50.5% at respective concentrations of 20, 40, 80, 160, 320, 640, and 1,280 μg/ml.

4. Conclusion: The test samples exhibited concentration-dependent ROS scavenging activity in the concentration range of 5 to 160 μg/ml, and were able to promote cell proliferation somewhat without any toxicity in normal cells but inhibited cell proliferation in a concentration-dependent manner without serious toxicity in colon cancer cells.

Through the above steps, the method for preparing the herbal composition having atopic immune system strengthening and anticancer effects according to embodiments of the present invention is completed. Also, the composition obtained by the above method of the present invention is harmless to human bodies, improves the functioning of the body, and activates blood circulation to thus maximize the efficient circulation of blood so as to improve the physical constitution and reinforce the physical strength, thereby solving the basic problems of atopy, and thus, the herbal composition of the invention, having atopic immune system strengthening and anticancer effects, may be provided in the form of a liquid, such as an herbal drink or an herbal yogurt.

### EXAMPLE 3

### Effect of Herbal Composition of the Present Invention on Amelioration of Atopy

In order to evaluate the amelioration effect by the herbal composition according to the present invention, the herbal composition was administrated two times a day for a total of 20 days to 8 male and female subjects with atopic dermatitis. After 1 month, the amelioration effect was evaluated and numbered into four steps, including very effective, slightly effective, no effective, and exacerbated.

TABLE 2
Herbal composition of the present invention

| Level of | Number of | |
|---|---|---|
| Effectiveness | subjects | Percentage |
| Very effective | 4 | 50.0% |
| Slightly effective | 3 | 37.5% |
| Not effective | 1 | 12.5% |
| Exacerbating | 0 | 0% |

The results of individual percentages are shown in Table 2. As shown in Table 2, 7 subjects among a total of 8 subjects using the herbal composition exhibited very effective and slightly effective amelioration effects. The proportion thereof is represented by 87.5%, which is regarded as showing a significant effect. For 1 month after use of the herbal composition of the invention in any one subject among “very effective” subjects, atopic dermatitis, accompanied by itching of the face, arms or legs, was considerably reduced after use of the herbal composition according to the present invention, and most of the lesions disappeared.

In order to aid the understanding of the present invention, the efficacies of individual herbal components are briefly described below, with reference to the literature.

\textcircled{1} Siberian ginseng (Acanthopanax senticosus) is a bush that grows in the deep valleys around Korea and loses its leaves every year, and has a height of 2 to 3 m. It is very densely covered with thin and long thorns and is gray-brown colored, and many thorns are attached to the bottom of the petiole. Siberian ginseng has palm-shaped compound leaves that are arranged alternately on the stem, and 3 to 5 small leaves with an inverted egg shape or a long oval shape. The surface of the leaves is sparsely hairy and the rear surface of young leaves has brown hair along the veins thereof and a sharp saw-toothed shape at the edge thereof. The petiole is as high as 3 to 8 cm and has many thorns. Its pale violet-like yellow flowers bloom in July, and it has an umbel in which flowers are attached to respective ends of branches or are parted at the bottom thereof. A honey gland is located at the position where the peduncle is parted. The style has a length of 1 to 1.8 cm and is completely combined, and the stigma is very shallowly divided into five parts. Its fruit is round, has a diameter of 8 to 10 mm, and ripens to black in September.

About 35 kinds of Acanthopanax plants grow all over the world, and 7 kinds, namely Acanthopanax sessiliflorus, Acanthopanax koreanum, Acanthopanax seoulense, Acanthopanax rufinerve, Acanthopanax rufinerve, Acanthopanax senticosus, and Acanthopanax senticosus var. koreanus grow freely in Korea, and any of them has been used to treat paralysis or weakness in folk remedies or herbal remedies.

The shape thereof is similar to that of wild ginseng of the same family Araliaceae. The Korean name “oga” means a five-leaved plant, like wild ginseng. As the representation of “ogapi”, corresponding to Acanthopanax, has been used for a long while, it is also called “ogalpi”. The palm-shaped leaves thereof resemble those of wild ginseng, and its ecological properties are also similar in that it grows in shady humus-rich soil deep in the mountains. The shape or properties of wild ginseng and Acanthopanax trees are similar to each other, with exception that wild ginseng is an herbaceous plant and Acanthopanax trees are woody.

Acanthopanax wine, made by peeling off Acanthopanax bark, is known to be very effective at treating back pain, limb numbness, partial paralysis and the like. Among various kinds of Acanthopanax trees, Acanthopanax senticosus, called Siberian ginseng, has been revealed to have the greatest efficacy. Siberian ginseng has been receiving worldwide attention recently since it was first spotlighted as a “natural drug having miracle efficacy” by scholars from the former Soviet Union.

Siberian ginseng having excellent efficacy is not easily observed in the wild except for the deep valleys of Kangwon Province or Mountain Baekdu, but is only visible in cultivated fields. Although it is reported to grow freely in deep valleys at a height 1000 m or more above sea level, it is difficult to find in practice.

Acanthopanax, which is the genus name of Siberian ginseng, means a “bramble for the treatment of all diseases” because “Acantho” indicates “bramble” and “panax” indicates treatment of all diseases. The term “panax” is a composite Greek word, pan meaning “all” and axos “cure”, denoting a “cure-all”, and Siberian ginseng is called “gasiogalpi” in Korea. All of the root, stem, leaves, fruits, and flowers thereof may be medicinally used (see Doosan Encyclopedia).

\textcircled{2} Fish mint (Houttuynia cordata) is the aerial part in flowering time of Houttuynia cordata Thunberg of Saururaceae in Korea, and means a fishy-smelling herb, so dubbed because of the fish smells emanating from the leaves thereof. In the Chinese Jin dynasty, it is referred to by the Chinese name “zuzi”, meaning a plant having a salted fish-like smell, of which “zu” indicates a juice.

This herb smells fishy when its leaves are rubbed and crushed, and has a spicy taste and slightly cold properties.

Fish mint has high antipyretic and drainage activities and is used for the treatment of coughing due to lung abscess, vomiting of bloody pus, pneumonia, acute or chronic bronchitis, gastroenteritis, urinary tract infections, and boils, and is also employed to relieve high fever or urinary difficulties.

The pharmacological effects thereof are reported to include antibacterial effects, immune-enhancing effects, anti-inflammatory effects, diuretic effects, and cough-inhibitory effects.

Its stem has vertical wrinkles and nodes. The lower nodes of the stem have fine radicles and are tender and easy to fold. The leaves thereof are dry or crumpled but are heart-shaped upon spreading. The leaves have pointed ends, and the upper surface thereof is dark yellowish green or brown and the lower surface thereof is gray-green or gray-brown (see Doosan Encyclopedia).

\textcircled{3} Manchurian wild rice (Zizania latifolia) is referred to by the Korean names “jingo”, and “gojangcho” or “go” in herbalism, and is recently receiving attention due to the increased attention to healthy food.

Zizania latifolia Tureg is a perennial plant belonging to Poaceae living in groups on the waterfront, in which “Zizania” is the barley field plant derived from the Greek word zizanion and “latifolia” means broad leaf.

It was used as a hardy wild plant capable of being used as food in a famine. The seeds thereof, referred to by the Korean name “gomi”, are eaten together with the plumules, and the leaves thereof are used to make rain capes, mats, blinds, etc.

In “Bonchogangmok” literature, the roots of Zizania latifolia facilitate urination and evacuation (but appropriately control the frequent urination due to diabetes), heal chronic disease of the bowels and the stomach, cure the aftereffects of drinking, aid digestion, and prevent thirst.

The culms of Zizania latifolia infected by the smut fungus (Ustilago edlilis) of the wheat or barley, called the Korean name “gosun” or “aebaek”, are used for Chinese food. It is reported to have efficacies including antipyretic effects, enhancement of gastroenteric functions, stopping of thirst, and treatment of red face, yellow eye, five visceral diseases, paralysis, and chest pain.

The soft-cored stem, resulting from peeling off the outer shells of the stem near the root, called “gosu” in Korean, functions to strengthen the teeth, stop the thirst, and treat diarrhea.

Such efficacies are deemed to treat hypertension, diabetes, hepatitis, gastrointestinal diseases, kidney disease, constipation, and skin diseases at present.

\textcircled{4} Dried ginger (Zingiber officinale Roscoe) is the dry rhizome of Zingiber officinale Roscoe in Korea, China, and Japan. It is called “kanshouga” in Japan and is used as ginger powder in Korea.

Its origin is deemed to be the Hindustan region of East India and it was reported to be cultivated about 2,500 years ago in China. The Sichuan province in China is known as the origin of ginger. In Korea, the cultivation of ginger was first reported in the book “Goryeosa” (in 1018, the ninth year of King Hyeonjong of Goryeo Dynasty, Korea), and moreover, ginger was reported to serve as the bounty of the king. According to one report, Shin Man-seok, who went as the envoy to China 1,300 years ago, brought back ginger and cultivated it in the Bongong region of Wanju gun, Korea, which is regarded as the start of ginger cultivation in Korea.

Ginger was described in the Chinese medical book about two thousand years ago, and has been utilized as a medicament in almost all herbal prescriptions. In daily life, it is widely useful as a flavoring material that reduces bad smells or realizes tasty cooking.

It has unique smells and is spicy and hot.

It is effective at treating small pain of the chest and the abdomen due to chills, digestive difficulties due to a cold abdomen, vomiting, and diarrhea. Also, ginger is used to treat weak pulse, cold and tingling limbs, chronic cough due to cold wind, shortness of breath, dysentery, and diarrhea.

Its pharmacological effects are promoting gastric secretion, activation of peristalsis of the intestinal tract, accelerating digestion, reducing vomiting, increasing blood pressure due to heart excitation, and promoting blood circulation. Anti-inflammatory, analgesic, and antibacterial actions are also provided.

Ginger is composed of a non-uniformly parted rhizome and pieces thereof, and is provided in the form of a slightly pressed flat or curved oval or a long oval with swollen lumps at opposite ends. The outer shell thereof is gray-yellow colored, and the outer surface thereof is off-white and has white spots thereon. Secretions are distributed as dark brown spots at the curved portions thereof, and essential oil-based yellow materials are observed using a microscope.

Dried ginger is also referred to as Zingiberis rhizoma or Amomum zingiber, and Zingiberis rhizoma Carbonisatum, as charred ginger is used for hemostasis (see Doosan Encyclopedia).

\textcircled{5} Cinnamon bark (Cinnamomum cassia) is the bark of Cinnamomum cassia Blume of Lauraceae in Korea and China.

Cinnamomum cassia Blume is an evergreen tree originating in southern China and is acquired between summer and fall and dried in the sun before use. The veins of Cinnamomum cassia Blume consist of a main center vein and two side veins, and thus the Chinese character “gui” is used for the plant name. The shape of the Chinese character “gui” resembles a fan-shaped tool on which the sequences of ancestor-memorial rites were written, called “holgi” in Korean, which shows various branches of the veins. Also, there is a historical case in which the Chinese character “gui”, indicative of an elongated pointed tablet of jade held in the hands by ancient rulers, was employed in the expression of Cinnamomum cassia Blume for all drugs. Cinnamomum cassia Blume may be referred to by the Korean name “chim”, meaning invasion, because it may invade and injure other trees and may also inhibit the growth of other plants. When this tree is nailed to the root of the other tree, the other tree is said to be certain to die.

This herb has a unique flavor, a spicy and sweet taste, and hot properties.

Cinnamon bark is used to treat the lower part of one's belly when cool, weakness of the waist and knees, lack of energy, frequent urination, dilute urine and excrement, diarrhea, vomiting, menstrual cramps, dysmenorrhea, postpartum abdominal pain due to stagnated blood, boils, skin ulcers, pain in limb paralysis, etc.

Its pharmacological effects are reported to include central nervous system depression, sedation, analgesic effects, antipyretic effects, increases in coronary blood flow, and inhibitory activity on skin fungal infection.

It is obtained in the form of a plate or a plate both ends of which are curled inwards, and the outer surface thereof is gray-brown colored and has rough and fine vertical wrinkles and small hollows protruding from both sides thereof. Off-white spots are sparsely distributed, and the inner surface thereof is red or brown colored.

\textcircled{6} Jujube (Ziziphus jujuba) is the fruit of a jujube tree, and the Chinese and Korean names therefor are “zao” and “mokmil”, respectively. The surface thereof is red-brown colored, and it has an oval shape and a length of 1.5 to 2.5 cm and tastes sweet when ripe. The fruit is eaten raw or is completely dried and used as a confection or for cooking and medicinal use. Jujubes are processed in daily life and are thus utilized as jujube wine, jujube tea, jujube vinegar, jujube soup, etc. In herbal use, diuretic, tonic and laxative activities are exhibited.

\textcircled{7} Citrus peel (Citrus unshiu) is the bark of Citrus unshiu Markovich of Rutaceae in Korea or the mature shell of a closely related plant. The fruit shells of citrus trees are medicinally used, and its taste is bitter and spicy, with hot properties. It is used to treat vomiting, nausea, or dyspepsia due to weakness of the spleen and stomach.

Citrus peel is regarded as improving with age. As its red color deepens, it is evaluated to be good, and thus is known as “reddish peel” or “Citrus unshiu peel”. The fruit is “Citrus unshiu Markovich”, the leaf is “Citrus tangerina Hort. et Tanaka”, the yellow portion of fruit peel without the core is “Citrus grandis Osbeck”, the seed is “Citrus reticulata Blanco”, and the unripe fruit peel is “Citrus reticulata”.

This herb has a unique smell, and is slightly pungent, spicy, bitter, and warm.

Citrus peel facilitates the circulation of energy and strengthens the functions of the spleen and is thus used to treat abdominal distention, belching, vomiting, nausea, indigestion, flatulence, drowsiness, and dilute excrement. Chronic coughing and phlegm are eliminated and diuretic activity is exhibited. Euphorbia fischeriana Steudel, Ephedra sinica Stapf, citrus peel, Evodia rutaecarpa Bentham, Pinellia ternata, and Poncirus trifolata are herbs, the efficacy of which is increased over time.

The essential oil component thereof is reported to have pharmacological effects, including digestive stimulation, digestive promotion, phlegm removal, anti-ulcer, anti-gastric secretion, cardiac action, blood pressure elevation, anti-allergy, promotion of bile secretion, uterine smooth muscle inhibition, antibacterial activity, etc.

It is obtained in irregular form, the outer surface of which is yellowish-red or dark tan and has many small recesses, and the inner surface of which is white or pale gray-brown, and it is lightweight and tends to crumble.

As described above, the herbal composition including seven components, namely Siberian ginseng, fish mint,

Manchurian wild rice, dried ginger, cinnamon bark, jujube and citrus peel, obtained by the above method of the present invention, can exhibit atopic immune system strengthening and anticancer effects.

Although the preferred embodiments of the present invention regarding the method for preparing the herbal composition having atopic immune system strengthening and anticancer effects have been disclosed for illustrative purposes, those skilled in the art will appreciate that various modifications, additions, deletion and substitutions are possible, without departing from the scope and spirit of the invention as disclosed in the accompanying claims.

1. A method for preparing an herbal composition having atopic immune system strengthening and anticancer effects, comprising:
providing an herbal mixture comprising 14 to 20 parts by weight of Siberian ginseng, 14 to 20 parts by weight of fish mint, 5 to 12 parts by weight of Manchurian wild rice, 14 to 20 parts by weight of dried ginger, 5 to 12 parts by weight of cinnamon bark, 14 to 20 parts by weight of jujube, and 14 to 20 parts by weight of citrus peel through water washing (S100); and
adding the herbal mixture with water at a ratio of 1:9 to 12 and heating the herbal mixture at 100 to 120° C. for 4 to 5 hours so as to carry out hydrothermal extraction (S200).

2. The method of claim 1, further comprising:
concentrating a hydrothermal extract from the hydrothermal extraction (S200) in a vacuum, thus obtaining a distillate (S300).

3. The method of claim 2, further comprising:
mixing the distillate with milk at a ratio of 1:3 to give a mixture that is then fermented using Lactobacillus, thus preparing a yogurt (S400);
adding blueberry and sugar at a ratio of 1:1 with a predetermined amount of water and performing heating for 30 minutes to 1 hour, thus preparing a jam (S500); and
adding the yogurt (S400) with the jam (S500), thus yielding a sweet yogurt (S600).
